SEQUENCE LISTING (11 sequences)

SEQ ID NO: 1 - DNA, 30 bp, Artificial Sequence
  synthetic target oligonucleotide nucleic acid
cattccgtagaatccagggaagcgtgtcac

SEQ ID NO: 2 - DNA, 36 bp, Artificial Sequence
  synthetic complementary capture probe for synthetic target oligonucleotide nucleic acid
aaaaaagtgacacgcttccctggattctacggaatc

SEQ ID NO: 3 - DNA, 36 bp, Artificial Sequence
  synthetic non-complementary capture probe for control biosensor
aaaaaacctctcgcgagtcaacagaatgcttaacat

SEQ ID NO: 4 - DNA, 26 bp, Artificial Sequence
  synthetic fully complementary capture probe 1 for mouse TP53 cDNA
aaaaaaatggaggattcacagtcgga

SEQ ID NO: 5 - DNA, 26 bp, Artificial Sequence
  synthetic fully complementary capture probe 2 for mouse TP53 cDNA
aaaaaatcagtctgagtcaggcccca

SEQ ID NO: 6 - DNA, 26 bp, Artificial Sequence
  synthetic capture probe 1 with single mismatched base for mouse TP53 cDNA
aaaaaatcagactgagtcaggcccca

SEQ ID NO: 7 - DNA, 26 bp, Artificial Sequence
  synthetic capture probe 2 with single mismatched base for mouse TP53 cDNA
aaaaaatcagtctgagtcacgcccca

SEQ ID NO: 8 - DNA, 26 bp, Artificial Sequence
  synthetic capture probe 3 with single mismatched base for mouse TP53 cDNA
aaaaaatcagtctcagtcaggcccca

SEQ ID NO: 9 - DNA, 26 bp, Artificial Sequence
  synthetic capture probe 4 with single mismatched base for mouse TP53 cDNA
aaaaaaatggtggattcacagtcgga

SEQ ID NO: 10 - DNA, 26 bp, Artificial Sequence
  synthetic capture probe 5 with single mismatched base for mouse TP53 cDNA
aaaaaaatggaggattcacagtcgga

SEQ ID NO: 11 - DNA, 26 bp, Artificial Sequence
  synthetic capture probe 6 with single mismatched base for mouse TP53 cDNA
aaaaaaatggaggatacacagtcgga

(19) United States
(12) Patent Application Publication
(10) Pub. No.: US 2008/0318232 A1
(43) Pub. Date: Dec. 25, 2008

(54) Nucleic Acid Biosensor with Photoelectrochemical Amplification

(75) Inventor: Zhiqiang Gao, Singapore (SG)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Agency For Science, Technology and Research, Centros #07-01 (SG)

(21) Appl. No.: 11/994,808

(22) PCT Filed: Jul. 5, 2005
(86) PCT No.: PCT/SG2005/000219

§ 371 Date: Aug. 5, 2008

(51) Int. Cl. C12Q 1/68

(52) U.S. Cl. 435/6; 204/666

(57) ABSTRACT

The present invention relates to electrode systems, methods, apparatus and chips for ultrasensitive detection and quantification of nucleic acids using photoelectrochemical amplification. Upon hybridization of a target nucleic acid to a nucleic acid capture probe, a photoreporter comprising a threading bis-intercalator selectively binds to double-stranded nucleic acid. The stability and reversibility of the photoreporter binding activity provides for ultrasensitive detection of nucleic acid hybridization events.

### TECHNICAL FIELD

The present invention relates to electrode systems, methods, apparatus and chips for ultra-sensitive detection and quantification of nucleic acids using photoelectrochemical amplification via binding of a photoreporter to a double-stranded nucleic acid analyte complex.

### BACKGROUND OF THE INVENTION

The use of biosensors to study gene expression has traditionally involved the use of labeled cDNA or cRNA targets derived from the mRNA of an experimental sample which are hybridized to nucleic acid capture probes attached to a solid support. By monitoring the amount of label associated with each hybridized event, it was possible to infer the abundance of each mRNA species represented. Although hybridization has been used for some time to detect and quantify nucleic acids, the combination of the miniaturization of the technology and the large and growing amounts of sequence information have enormously expanded the scale at which gene expression can be studied.

Over the past decade, there have been significant advances in the development of nucleic acid biosensors based on the immobilization of short oligonucleotide capture probes onto a solid support, which can then be used as biorecognition elements upon subsequent hybridization with target sample nucleic acids. The most popular methods remain those relying on the use of fluorescently-conjugated target nucleic acid¹.

However, adequate sensitivity for detection of low copy number genes has remained problematic in the application of current fluorescence-based microarray technology. Most contemporary fluorescent microarray assays are performed in conjunction with solution phase (off-chip) pre-amplification and/or labeling approaches such as polymerase chain reaction (PCR).² However, PCR amplification not only prolongs assay time but also can often introduce contaminating amplicon species. In addition, genes may not be represented in relatively proportional levels in the final PCR product as compared to the initial sample due to selective and nonlinear target amplification.³ Furthermore, the incomplete denaturation of nucleic acid secondary structure during cDNA synthesis can also compromise polymerase activity, resulting in truncated cDNA copies of target genes. Moreover, PCR-based pre-amplification steps are of limited application in analyzing nucleic acids of high complexity, as the products of such PCRs may interfere with each other, thereby resulting in a loss of amplification efficiency and specificity⁴. Off-chip target pre-amplification approaches also significantly increase the cost of biosensor procedures and often lead to sequence-dependent quantification bias.

In order to address these technical difficulties, several on-chip amplification strategies such as rolling circle amplification⁴, branched DNA technology⁵, catalyzed reporter deposition⁶, dendritic tags⁷, enzymatic amplification^{8,9}, and chemical amplification^{10,11} have been proposed. Among these, the amplification strategies coupled with electronic transduction methods have the greatest potential to provide a simple, accurate, and inexpensive platform for nucleic acid assays due to inherent miniaturization of electronic devices and their compatibility with advanced semiconductor technologies.

The present invention is predicated on the surprising and unexpected finding by the inventors that the threading bis-intercalator, PIND-Ru-PIND, can function as a highly sensitive, highly stable and highly selective photoreporter, enabling photoelectrochemical detection of nucleic acid hybridization events.

### SUMMARY OF THE INVENTION

According to a first aspect of the present invention, there is provided an electrode system, wherein said electrode system comprises:
(a) a working electrode;
(b) a nucleic acid capture probe coupled to the working electrode, said probe comprising a sequence complimentary to a sequence of a target nucleic acid; and
(c) a threading PIND-Ru-PIND bis-intercalator
   such that said target nucleic acid is hybridized to said nucleic acid capture probe to form a double-stranded nucleic acid complex, and said complex is intercalated with said threading PIND-Ru-PIND bis-intercalator. The amount of said intercalated PIND-Ru-PIND is indicative of the amount of target nucleic acid. The electrode system may be for detecting and/or quantifying the target nucleic acid.

The electrode system may further comprise a support onto which is disposed the working electrode.

The working electrode may comprise an array of said nucleic acid capture probes.

The working electrode may comprise at least one of diamond, glassy carbon, gold, graphite, indium tin oxide, platinum or silicon. The working electrode may comprise indium tin oxide.

The nucleic acid capture probe may comprise DNA. Additionally or alternatively, the nucleic acid capture probe may comprise RNA. The nucleic acid capture probe may be attached to the working electrode.

The target nucleic acid may comprise DNA. The DNA may comprise cDNA. Additionally or alternatively, the target nucleic acid may comprise RNA. The RNA may comprise cRNA.

The electrode system may further comprise a reference electrode and a counter electrode.

In one embodiment of the first aspect, the intercalation of the double-stranded nucleic acid complex with the threading PIND-Ru-PIND bis-intercalator may involve intercalation of two naphthalene diimide groups from the threading PIND-Ru-PIND bis-intercalator with the double-stranded nucleic acid complex, thus forming an ion pair between a phosphate of the double-stranded nucleic acid complex and a bicationic Ru(bpy)₂²⁺ group of the threading PIND-Ru-PIND bis-intercalator. The stable adduct thereby formed may produce a photoelectrochemical response to exposure to a light source, producing a photocurrent action spectra in the range of 400-600 nm, and in particular at 490 nm. The application of illumination cycles may cause a photocharging and discharging current to increase substantially linearly with increasing number of said illumination cycles, up to 10³ cycles. The stability and sensitivity of the intercalated threading PIND-Ru-PIND bis-intercalator may provide for the detection of target nucleic acid up to a dilution of 1×10⁻¹⁶ M target nucleic acid.

According to a second aspect of the present invention, there is provided a method for detecting a target nucleic acid, wherein said method comprises:
(a) hybridizing said target nucleic acid with a nucleic acid capture probe coupled to a working electrode, forming a double-stranded nucleic acid complex;
(b) intercalating said double-stranded nucleic acid complex with a threading PIND-Ru-PIND bis-intercalator;
(C) illuminating the working electrode with light comprising at least one wavelength that photoactivates the PIND-Ru-PIND bis-intercalator;
(d) applying a potential to the working electrode; and
(e) obtaining a photocurrent action spectrum.

The amount of said target nucleic acid may be determined from said photocurrent action spectrum.

According to a third aspect of the present invention, there is provided a method for quantifying a target nucleic acid, wherein said method comprises:
(a) hybridizing said target nucleic acid with a nucleic acid capture probe coupled to a working electrode, forming a double-stranded nucleic acid complex;
(b) intercalating said double-stranded nucleic acid complex with a threading PIND-Ru-PIND bis-intercalator;
(c) illuminating the working electrode with light comprising at least one wavelength that photoactivates the PIND-Ru-PIND bis-intercalator;
(d) applying a potential to the working electrode; and
(e) obtaining a photocurrent action spectrum.
   The amount of said target nucleic acid may be determined from said photocurrent action spectrum.

According to a fourth aspect of the present invention, there is provided an apparatus for detecting a target nucleic acid, wherein said apparatus comprises:
(a) the electrode system of the first aspect;
(b) means for illuminating the working electrode of the electrode system;
(c) means for applying a potential to the working electrode; and
(d) means for obtaining a photocurrent action spectrum.

In use, illumination of the working electrode of the electrode system by the means for illuminating the working electrode, and application of a potential to the working electrode by the means for applying a potential to the working electrode, generates a photocurrent action spectrum, which is obtained by the means for obtaining a photocurrent action spectrum. The amount of said target nucleic acid may be determined from said photocurrent action spectrum.

The apparatus may further comprise a support onto which is disposed the working electrode.

The apparatus may optionally further comprise a data capture device. The data capture device may provide a means for applying a potential to the working electrode. The data capture device may provide a means for obtaining a photocurrent action spectrum of the working electrode.

The apparatus may further comprise a fluid system. The fluid system may provide a means for delivering the target nucleic acid to the electrode system.

The apparatus may further provide a temperature control device. The temperature control device may be used in conjunction with the fluid system.

The apparatus may further comprise an optical scanner or detector. The optical scanner or detector may receive data from the electrode system. The data may be sample identifiers. Additionally or alternatively, the data may be fluorescence data.

According to a fifth aspect of the present invention, there is provided an apparatus for quantifying a target nucleic acid, wherein said apparatus comprises:
(a) the electrode system of the first aspect;
(b) means for illuminating the working electrode of the electrode system;
(c) means for applying a potential to the working electrode; and
(d) means for obtaining a photocurrent action spectrum.

In use, illumination of the working electrode of the electrode system by the means for illuminating the working electrode, and application of a potential to the working electrode by the means for applying a potential to the working electrode, generates a photocurrent action spectrum, which is obtained by the means for obtaining a photocurrent action spectrum. The amount of said target nucleic acid may be determined from said photocurrent action spectrum.

The apparatus may further comprise a support onto which is disposed the working electrode.

The apparatus may optionally further comprise a data capture device. The data capture device may provide a means for applying a potential to the working electrode. The data capture device may provide a means for obtaining a photocurrent action spectrum of the working electrode.

The apparatus may further comprise a fluid system. The fluid system may provide a means for delivering the target nucleic acid to the electrode system.

The apparatus may further provide a temperature control device. The temperature control device may be used in conjunction with the fluid system.

The apparatus may further comprise an optical scanner or detector. The optical scanner or detector may receive data from the electrode system. The data may be sample identifiers, Additionally or alternatively, the data may be fluorescence data.

According to a sixth aspect of the present invention, there is provided a biosensor chip comprising:
(a) a chip;
(b) at least one working electrode disposed on the chip;
(c) a nucleic acid capture probe coupled to the working electrode, said probe comprising a sequence complimentary to a sequence of a target nucleic acid; and
(d) a threading PIND-Ru-PIND bis-intercalator.

The chip may comprise the working electrode.

The working electrode may comprise an array of said nucleic acid capture probes.

The working electrode may comprise at least one of diamond, glassy carbon, gold, graphite, indium fin oxide, platinum or silicon. The working electrode may comprise indium tin oxide.

The nucleic acid capture probe may comprise DNA. Additionally or alternatively, the nucleic acid capture probe may comprise RNA.

The target nucleic acid may comprise DNA. The DNA may comprise cDNA. Additionally or alternatively, the target nucleic acid may comprise RNA. The RNA may comprise cRNA.

### DEFINITIONS

In the context of this specification, the term “comprising” means “including principally, but not necessarily solely”. Furthermore, variations of the word “comprising”, such as “comprise” and “comprises”, have correspondingly varied meanings.

The term “primer” is used herein interchangeably with the term “oligonucleotide”. The term “primer” means a single-stranded oligonucleotide capable of acting as a point of initiation of template-directed DNA synthesis. An “oligonucleotide” is a single-stranded nucleic acid typically ranging in length from 2 to about 500 bases. The precise length of an oligonucleotide will vary according to the particular application, but typically ranges from 15 to 30 nucleotides. An oligonucleotide need not reflect the exact sequence of the template but must be sufficiently complementary to hybridize to the template.

As used herein the term “hybridize” means, in the context of nucleic acids, to form base pairs between complementary regions of two nucleic acids that were not originally paired.

As used herein, the term “chip” is used interchangeably with the terms “array” or “microarray” and refers to an apparatus comprising, or onto which is disposed, a working electrode. Typically a nucleic acid capture probe is coupled to the working electrode.

### BRIEF DESCRIPTION OF THE DRAWINGS

FIG. 1. Schematic diagram of one embodiment of the present invention.

FIG. 2. (A) Cyclic voltammograms of a 50 nM of target nucleic acid hybridized and PIND-Ru-PIND intercalated biosensor, (1) first scan and (2) third scan, and (3) a control biosensor. Potential scan rate 100 mV/s, (B) Photoelectrochemical responses: (1) the 25 nM target nucleic acid hybridized and PIND-Ru-PIND intercalated biosensor; and (2) control biosensor. Insert: Stability test of the biosensor. Wavelength 490 nm, light intensity 22.4 mW/cm², applied potential 0.10 V.

FIG. 3. Photocurrent action spectra of (1) a control biosensor, (2) a 1.0 nM target nucleic acid hybridized and PIND-Ru-PIND treated biosensor, and (3) UV-vis adsorption spectrum of 25 μM PIND-Ru-PIND in H₂O. Illumination was conducted with monochromatic light at a 10-nm interval. The photocurrent was collected at 0.10 V.

FIG. 4. Dependence of photocurrent on the incident light intensity (400-600 nm) of a 1.0 nM nucleic acid hybridized and PIND-Ru-PIND intercalated biosensor at 0.10 V.

FIG. 5. Dependence of photocurrent on the applied potential of a 1.0 nM target DNA hybridized and PIND-Ru-PIND treated complementary capture probe coated electrode. Illumination was conducted with a 23.1 mW/cm² monochromatic light beam of 490 nm.

FIG. 6. Photoelectrochemical response of multiple illumination cycles of a 100 pM target nucleic acid hybridized and PIND-Ru-PIND treated biosensor. Wavelength 490 nm, light intensity 22.8 mW/cm².

FIG. 7. Photoelectrochemical responses of TP53 mouse cDNA at different concentrations. Integration of 1000 cycles, wavelength 490 nm, light intensity 22.7 mW/cm². Insert: Photoelectrochemical responses at low concentration end.

FIG. 8. Photoelectrochemical responses of biosensors after hybridization and intercalation in 50 ng mRNA mixture with capture probes (1) and (3) complementary, and (2) one-base mismatch to mouse TP53. Wavelength 490 nm, light intensity 22.9 mW/cm².

### BEST MODE OF PERFORMING THE INVENTION

The threading bis-intercalator, PIND-Ru-PIND, where:

PIND=N,N′-bis(3-propyl-imidazole)-1,4,5,8-naphthalene diimide

Ru=Ru(bpy)₂²⁺(bpy=2,2′-bipyridine)

was employed in experiments demonstrating the ultrasensitive non-labeling detection of nucleic acid hybridization events. A remarkable sensitivity enhancement was achieved compared to direct voltammetric detection. A photoelectrochemical signal was observed when as little as femptomolar (10⁻¹⁵ M) amounts of nucleic acid were present, representing a 10⁴-fold increase in the sensitivity of detection of nucleic acids over conventional techniques. Thus the present invention offers an improved approach to hybridization-based nucleic acid biosensor applications.

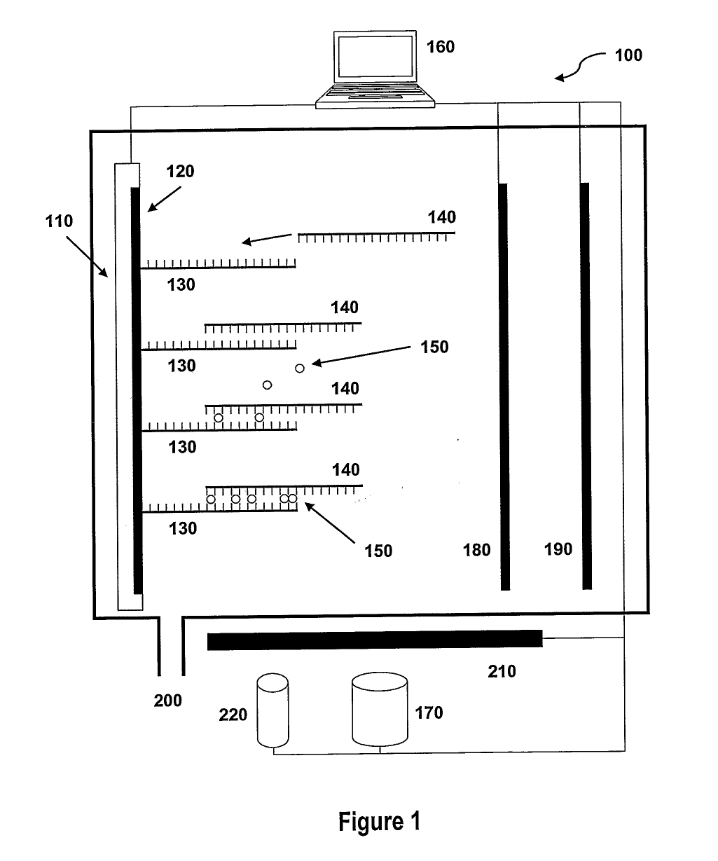

FIG. 1 illustrates one embodiment of the invention. A system (100) comprises a support (110), a working electrode (120) disposed on the support (110), a data capture device (160), a light source (170), and optionally a counter electrode (180), a reference electrode (190), a fluid system (200), a temperature control device (210) and an optical scanner or detector (220). A nucleic acid capture probe (130) is coupled to the working electrode (120) and composes a sequence complimentary to a sequence of a target nucleic acid (140). Upon hybridization of the target nucleic acid (140) with the nucleic acid capture probe (130) to form a double-stranded nucleic acid complex, the complex is then intercalated with a threading PIND-Ru-PIND bis-intercalator (150), the amount of the intercalated PIND-Ru-PIND (150) being indicative of the amount of target nucleic acid (140).

The support (110) can comprise any material and be of any shape, size, dimension, density and/or conductibility as required for performance of the present invention. In some embodiments, the support (110) and/or the working electrode (120) comprises a means for determining, in conjunction with the optical scanner or detector (220), the location on the working electrode (120) that the light source (170) is illuminating. For example, there may be provided an identifier such as a grid or other reference mark or indice for determining the illumination position. Such identifiers can be applied to the support (110) and/or the working electrode (120) by any method known in the art that enables performance of the invention, including but not limited to physical or chemical vapour deposition, lithography, ion-assisted or electrochemical etching or electroplating. In other embodiments, where the working electrode (120) comprises an array of nucleic acid capture probes (130), the identifier can comprise part of the array, such as a nucleic acid capture probe (130) or a coloured, fluorescent or photoelectrochemical molecule positioned within the array. Where the identifier is a nucleic acid capture probe (130), an identifier target nucleic acid (140) is added and hybridized to the identifier nucleic acid capture probe (130), thereby forming an identifying nucleic acid complex. Upon intercalation of the identifying nucleic acid complex with a threading PIND-Ru-PIND bis-intercalator (150), illumination of the complex by a light source (170) results in the generation of an identifying photoelectrochemical signal. In other embodiments, the support (110) comprises a means for generating an electrical signal, such as a photovoltaic, photoresistant or other light sensitive means, upon illumination from the light source (170), thereby identifying the location of the illumination.

The working electrode (120) is any electrode known in the art that is compatible with the disclosed assay, and may comprise at least one of diamond, glassy carbon, gold, graphite, indium tin oxide, platinum or silicon. Preferably the working electrode (120) comprises an indium tin oxide electrode. In some embodiments, the optical arrangement of the components may involve the working electrode (120) deposited as a layer on the support (110) using any means known in the art, for example, by printing, coating, or either physical or chemical vapour deposition. The working electrode (120) has any suitable physical shape, for example, rectangular, circular or any form of polygon. Furthermore, the working electrode (120) may either be planar or non-planar. Non-planar examples include any prism, such as a cylinder, cone or pyramid.

The nucleic acid capture probe (130) may comprise a nucleic acid of any form, for example, including but not limited to DNA, cDNA, gDNA, RNA, cRNA, tRNA, mRNA, rRNA, RNAi, iRNA, shRNA, PNA or LNA or any combination thereof. The nucleic acid capture probe (130) may be in any sequence designed to be of a sequence complimentary to the sequence of a target nucleic acid (140) so as provide for hybridization or annealing of the nucleic acid capture probe (130) with the target nucleic acid (140). In some embodiments, a control nucleic acid capture probe (130) may be of a sequence that is different by at least one nucleotide residue to the sequence of a target nucleic acid (140). The nucleic acid capture probe (130) is coupled to the working electrode (120). In some embodiments, the working electrode (130) is composed of indium tin oxide (ITO) and is pretreated by silanization, with nucleic acid capture probes (130) being aldehyde modified prior to immobilization on the working electrode (120). Preferably, an aliquot of denatured aldehyde modified nucleic acid capture probes (130) is dispensed onto the silanized working electrode (120) and incubated for a time of 2 to 3 hours at a temperature of 20° C. The working electrode (120) is then soaked in vigorously stirred hot water for a time of 2 minutes at a temperature in the range of 90° C. to 95° C. The working electrode (120) coated with the nucleic acid capture probe (130) is further immersed in an ethanolic solution for a period of up to 24 hours. Preferably, the period of immersion is in the range of 1 to 7 hours. Still more preferably, the immersion period is in the range of 3 to 5 hours. The surface density of the immobilized nucleic acid capture probes (130) on the working electrode (120) may be in the range of 5-8×10⁻¹² mol/cm². In other embodiments, the nucleic acid capture probe (130) is attached to the working electrode (120) using an attachment group, the identity of which will depend upon the composition of the working electrode (120). For example, attachment of a nucleic acid capture probe (130) to a gold electrode is well known in the art through the use of a thiol attachment group, attachment of a nucleic acid capture probe (130) to a diamond electrode is well known in the art through the use of a diazonium attachment group, attachment of a nucleic acid capture probe (130) to a silicon electrode is well known in the art through the use of a substituted alkoxysilane attachment group, attachment of a nucleic acid capture probe (130) to a glassy carbon electrode is well known in the art through the use of an amine or carbodiimide attachment group, and attachment of a nucleic acid capture probe (130) to other electrodes is well known in the art through the use of carboxylate-amine functional groups and biotin-avidin coupling.

The nucleic acid capture probe (130) is designed to hybridize to a target nucleic acid (140) of any sequence in any form, for example, including but not limited to DNA, cDNA, gDNA, RNA, cRNA, tRNA, mRNA, rRNA, RNAi, iRNA, shRNA, PNA or LNA or any combination thereof. The hybridization event can result in the formation of a DNA-DNA homoduplex, RNA-RNA homoduplex or DNA-RNA homoduplex or any other nucleic acid complex. The efficiency of hybridization of the target nucleic acid (140) with the nucleic acid capture probe (130) may be in a range including but not limited to 10%-75%, 20% to 40%, 25% to 35%, 27% to 35%, 30% to 35%, 31% to 35%, 31% to 34%, 31% to 33% or 30% to 33%. Preferably, the hybridization efficiency is approximately 32%, representing 10% of the target nucleic acid (140) hybridized to the nucleic acid capture probe (130).

The threading PIND-Ru-PIND bis-intercalator (150) is a photoelectrochemical intercalator that preferentially binds to double-stranded nucleic acid rather than single-stranded nucleic acid. In some embodiments, the intercalation of a double-stranded nucleic acid complex with the threading PIND-Ru-PIND bis-intercalator (150) forms a stable adduct by intercalation of two naphthalene diimide groups from the threading PIND-Ru-PIND bis-intercalator (150) with the double-stranded nucleic acid complex, forming an ion pair between a phosphate of the double-stranded nucleic acid complex and a bicationic Ru(bpy)₂²⁺ group of the threading PIND-Ru-PIND bis-intercalator (150).

Application of a voltage to the stable adduct comprising the intercalated threading PIND-Ru-PIND bis-intercalator (150) and the double-stranded nucleic acid complex may provide a steady-state cyclic voltammogram between the third and fifth cycles, the third and tenth cycles, the third and twentieth cycles, the third and fiftieth cycles or after the third cycle. The steady state voltammogram may be achieved at a potential scan rate of 100 mV/s when applying a voltage verses a Ag/AgCl electrode in a range including but not limited to 0-1.0V, 0.1-1.0V, 0.2-1.0V, 0.3-1.0V, 0.4-1.0V, 0.5-1.0V, 0.5-0.9V, 0.5-0.95V, 0.55-0.9V, 0.6-0.9V, 0.6-0.95V, 0.65-0.9V, 0.65-0.95V or 0.6-1.0V. This steady state voltammogram indicates the high stability of the threading PIND-Ru-PIND bis-intercalator (150) when intercalated with the double-stranded nucleic acid complex.

Evaluation of the intercalated threading PIND-Ru-PIND bis-intercalator (150) as a potential redox active indicator may provide a detection limit of 0.50 nM and a dynamic range including but not limited to 0.5-550 nM, 0.5-150 nM, 0.5-100 nM, 0.5-75 nM, 0.5-50 nM, 0.5-20 nM, 0.5-5 nM, 0.8-5 nM, 0.8-10 nM, 0.8-15 nM, 0.8-25 nM, 0.8-50 nM, 0.8-100 nM or 0.8-200 nM. The number of intercalated threading PIND-Ru-PIND bis-intercalator (150) molecules as determined by integration of oxidation or reduction current peak at a low scan rate may yield a charge in a range including but not limited to 0.1-0.5 μC, 0.25-0.35 μC, 0.25-0.34 μC, 0.25-0.33 μC, 0.25-0.32 μC, 0.25-0.31 μC, 0.25-0.30 μC, 0.26-0.35 μC, 0.27-0.35 μC, 0.27-0.35 μC, 0.28-0.35 μC, 0.27-0.31 μC or 0.28-0.30 μC. The ratio of intercalated threading PIND-Ru-PIND bis-intercalator (150) molecules to base pairs of the hybridized nucleic acid capture probe (130) target nucleic acid (140) complex may be in a range including but not limited to 1/1-1/20, 1/1-1/10, 1/2-1/10, 1/3-1/10, 1/4-1/10, 1/5-1/10, 1/3-1/7, 1/4-1/7, 1/5-1/7, 1/5-1/8, 1/4-1/6, 1/3-1/6 or 1/5-1/6.

The stable adduct produces a photoelectrochemical response to exposure to a light source, producing a photocurrent action spectra in the range of 400-600 nm, more preferably at 490 nm. The application of up to 10³ illumination cycles over the period of 60 minutes may cause a photocharging and discharging current to increase substantially linearly with increasing number of illumination cycles, with a time period for the current to drop from the maximum level to the background level in a range including but not limited to 1.5 to 5.0 seconds, 1.7 to 3.0 seconds, 1.7 to 2.0 seconds, 1.75 to 2 seconds, 1.8 to 2.0 seconds, 1.85 to 2.0 seconds, 1.75 to 2.1 seconds, 1.75 to 2.2 seconds, 1.75 to 2.3 seconds, 1.75 to 2.4 seconds, 1.75 to 2.5 seconds, 1.75 to 3.0 seconds, 1.8 to 2.1 seconds, 1.8 to 2.2 seconds, 1.8 to 2.3 seconds, 1.8 to 2.4 seconds, 1.8 to 2.5 seconds, 1.8 to 3.0 seconds, 1.85 to 2.0 seconds, 1.85 to 2.1 seconds, 1.85 to 2.2 seconds, 1.85 to 2.3 seconds, 1.85 to 2.4 seconds, 1.9 to 3.0 seconds, 1.9 to 2.5 seconds. The stability and sensitivity of the intercalated threading PIND-Ru-PIND bis-intercalator (150) may provide for the detection of target nucleic acid (140) diluted in a range including but not limited to 1.0M to 1×10⁻¹⁷M, 1.0M to 1×10⁻¹⁶M, 1.0M to 1×10⁻¹⁵M, 1×10⁻³M to 1×10⁻¹⁷M, 1×10⁻³M to 1×10⁻¹⁶M, 1×10⁻³M to 1×10⁻¹⁵M, 1×10⁻⁶M to 1×10⁻¹⁷M, 1×10⁻⁶M to 1×10⁻¹⁶M, 1×10⁶M to 1×10⁻¹⁵M, 1×10⁻⁹M to 1×10⁻¹⁷M, 1×10⁻⁹M to 1×10⁻¹⁶M to 1×10⁻⁹M to 1×10⁻¹⁵M, 1×10⁻¹²M to 1×10⁻¹⁷M, 1×10⁻¹²M to 1×10⁻¹⁶M, 1×10⁻¹²M to 1×10⁻¹⁵M, 1×10⁻¹³M to 1×10⁻¹⁷M, 1×10⁻¹³M to 1×10⁻¹⁶M, 1×10⁻¹³M to 1×10⁻¹⁵M, 1×10⁻¹⁴M to 1×10⁻¹⁷M, 1×10⁻¹⁴M to 1×10⁻¹⁶M, 1×10⁻¹⁴M to 1×10⁻¹⁵M, 1×10⁻¹⁰M to 1×10⁻¹⁶M, 1×10⁻¹¹M to 1×10⁻¹⁶M, 1×10⁻¹³M to 1×10⁻¹⁶M, 1×10⁻¹⁴M to 1×10⁻¹⁶M, 1×10⁻¹⁵M to 1×10⁻¹⁶M or 1×10⁻¹⁵M to 5×10⁻¹⁷M. Dilution and/or titration of target nucleic acid (140) may be required when using concentrations that may saturate the nucleic acid capture probe (130).

The photocharging/discharging current may increase linearly with the incident light intensity in a range including but not limited to 0.05 mW/cm² to 20.0 mW/cm², 0.05 mW/cm² to 19.9 mW/cm², 0.05 mW/cm² to 19.8 mW/cm², 0.05 mW/cm² to 19.7 mW/cm², 0.05 mW/cm² to 19.6 mW/cm², 0.05 mW/cm² to 19.5 mW/cm², 0.05 mW/cm² to 19.4 mW/cm², 0.05 mW/cm² to 19.3 mW/cm², 0.05 mW/cm² to 19.2 mW/cm², 0.05 mW/cm² to 19.1 mW/cm², 0.05 mW/cm² to 19.0 mW/cm², 1.0 mW/cm² to 20.0 mW/cm², 1.0 mW/cm² to 19.9 mW/cm², 1.0 mW/cm² to 19.8 mW/cm², 1.0 mW/cm² to 19.7 mW/cm², 1.0 mW/cm² to 19.6 mW/cm², 1.0 mW/cm² to 19.5 mW/cm², 1.0 mW/cm² to 19.4 mW/cm², 1.0 mW/cm² to 19.3 mW/cm², 1.0 mW/cm² to 19.2 mW/cm², 1.0 mW/cm² to 19.1 mW/cm², 1.0 mW/cm² to 19.0 mW/cm², 2.5 mW/cm² to 20.0 mW/cm², 2.5 mW/cm² to 19.9 mW/cm², 2.5 mW/cm² to 19.8 mW/cm², 2.5 mW/cm² to 19.7 mW/cm², 2.5 mW/cm² to 19.6 mW/cm², 2.5 mW/cm² to 19.5 mW/cm², 2.5 mW/cm² to 19.4 mW/cm², 2.5 mW/cm² to 19.3 mW/cm², 2.5 mW/cm² to 19.2 mW/cm², 2.5 mW/cm² to 19.1 mW/cm², 2.5 mW/cm² to 19.0 mW/cm², 2.6 mW/cm² to 20.0 mW/cm², 2.6 mW/cm² to 19.9 mW/cm², 2.6 mW/cm² to 19.8 mW/cm², 2.6 mW/cm² to 19.7 mW/cm², 2.6 mW/cm² to 19.6 mW/cm², 2.6 mW/cm² to 19.5 mW/cm², 2.6 mW/cm² to 19.4 mW/cm², 2.6 mW/cm² to 19.3 mW/cm², 2.6 mW/cm² to 19.2 mW/cm², 2.6 mW/cm² to 19.1 mW/cm², 2.6 mW/cm² to 19.0 mW/cm², 2.7 mW/cm² to 20.0 mW/cm², 2.7 mW/cm² to 19.9 mW/cm², 2.7 mW/cm² to 19.8 mW/cm², 2.7 mW/cm² to 19.7 mW/cm², 2.7 mW/cm² to 19.6 mW/cm², 2.7 mW/cm² to 19.5 mW/cm², 2.7 mW/cm² to 19.4 mW/cm², 2.7 mW/cm² to 19.3 mW/cm², 2.7 mW/cm² to 19.2 mW/cm², 2.7 mW/cm² to 19.1 mW/cm² or 2.7 mW/cm² to 19.0 mW/cm².

The threading PIND-Ru-PIND bis-intercalator (150) is believed to be in an “excited” state and more readily oxidized when it is intercalated into a double-stranded nucleic acid complex, than when it is in a non-intercalated “ground” state. Thus, biasing the working electrode (120) to a potential sufficient to oxidize the threading PIND-Ru-PIND bis-intercalator (150) when in an excited (intercalated) state but not when in a ground (non-intercalated) state results in the oxidization of only the intercalated threading PIND-Ru-PIND bis-intercalator (150). In some embodiments, the oxidized intercalated threading PIND-Ru-PIND bis-intercalator (150) remains bound to the double-stranded nucleic acid complex. In other embodiments, the oxidized intercalated threading PIND-Ru-PIND bis-intercalator (150) is reduced back to the ground state by a sacrificial reductant, thereby causing a photocurrent until either the sacrificial reductant is exhausted or the illumination is discontinued.

The data capture device (160) controls the system (100), including but not limited to functioning as or controlling a potentiostat for controlling and measuring the voltage and/or current in the system (100). The potentiostat may be any electronic device that controls the voltage difference between the working electrode (120) and the reference electrode (190). The potentiostat may implement this control by injecting current into the system (100) through a counter electrode (180). The potentiostat may measure the current flow between the working electrode (120) and the other electrodes. A controlled variable in the potentiostat may be the system potential and a measured variable may be the system current. The potentiostat may cause a control voltage to force a current through the counter electrode (180) exactly as high as to achieve the desired potential difference between working electrode (120) and reference electrode (190). The control voltage may be produced by the internal potential control source of the potentiostat, or by an external signal generator, for example, a ramp generator or a sine wave generator. The potentiostat may be used as controlled precision voltage source, whereby the potential that is fed into the system (100) (or set by the internal voltage source) may directly control the voltage of the counter electrode (180). The maximum current may be limited by the set current range, and by the power of the potentiostat, such that voltages applied beyond a range of a control voltage requires insertion of a potentiometric divider, thereby increasing the voltage amplification of the potentiostat. Alternatively, the potentiostat may function as a controlled current source or as a precision ammeter. In addition, potentiostats may be capable of measuring currents as small as single electrons, for example, using quantum dots, scanning probe devices and single electron tunneling devices. Photocurrent may be measured using a spectrofluorometer.

In some embodiments, the data capture device (160) regulates the light source (170), including but not limited to the portion or portions of the working electrode (120) illuminated. For example, the data capture device (160) can direct the light source (170) to scan the working electrode (120) in a predetermined pattern. This is useful in embodiments where the working electrode (120) comprises an array of nucleotide capture probes (130) coupled to the working electrode (120). In other embodiments, the data capture device (160) optionally receives data from an optical scanner or detector (220) that is configured to receive optical information from either the working electrode (120) or the support (110). For example, the optical information may comprise optically encoded sample identifiers, such as barcodes. Alternatively, the data capture device (160) is configured to receive sample identification data from either the working electrode (120) or the support (110) coded by other means, for example, using a radio frequency tag or other integrated device such as a microprocessor. In further embodiments, the data capture device (160) implements a particular operating procedure for the system (100) upon receipt of particular encoded sample identifying data. In yet further embodiments, the optical scanner or detector (220) receives fluorescence data from the working electrode (120) generated by a photoelectrochemical response elicited from the intercalated threading PIND-Ru-PIND bis-intercalator (150), with such fluorescence data then sent to the data capture device (160). In other embodiments, the data capture device (160) may acquire, process and respond to data from optional components, including but not limited to a counter electrode (180), a reference electrode (190), a fluid system (200), a temperature control device (210) and/or an optical scanner or detector (220). The data capture device (160) is illustrated as a single component but may comprise several components. These components may be specific to a particular function of the system (100), and may comprise modular components, thereby permitting a user the flexibility of adding or deleting specific components depending upon the desired functions of the system (100). The data capture device (160) may comprise at least one interface for data acquisition known in the art, for example, a display, keyboard or keypad, printer and/or peripheral data port. The data capture device (160) may be programmable by a user or pre-programmed, or a combination of both.

The light source (170) comprises any source of sufficient intensity and energy capable of eliciting a photoelectrochemical response from the threading PIND-Ru-PIND bis-intercalator (150). Suitable light sources include, but are not limited to, a laser, an arc lamp, a light emitting diode (LED), for example a blue light or a blue-green light emitting diode, a fluorescent lamp, a halogen lamp, a metal halide lamp, a discharge lamp, for example a xenon discharge lamp, a tungsten incandescent lamp, a high pressure sodium lamp or the sun. The light source chosen is one that emits light in at least around or at 490 nm, for example, in the range of about 400-600 nm, 410-590 nm, 420-580 nm, 430-570 nm, 440-560 nm, 450-550 nm, 460-540 nm n, 450-550 nm, 460-540 nm, 470-530 nm, 480-520 nm, 480-510 nm, 480-500 nm, 481-499 nm, 482-498 nm, 483-497 nm, 484-496 nm, 485-495 nm, 486-494 nm, 487-493 nm, 488-492 nm or 489-491 nm. The light may be infrared, visible or ultraviolet light. In some embodiments, the light source (170) is configured to direct electromagnetic radiation to the working electrode (120) or a portion thereof. In other embodiments, the light source (170) is configured to scan the surface of the working electrode (120) in a predetermined pattern. In some embodiments, the light source (170) scans the working electrode (120) by moving, or by the working electrode (120) moving. A laser is a particularly useful light source (170) for a system (100) comprising an array of nucleic acid capture probes (130) coupled to a working electrode (120).

The optional counter electrode (180) and reference electrode (190) are any type known in the art that are suitable for performance of the present invention. Preferably, the counter electrode (180) is a platinum wire and the reference electrode (190) is a silver/silver chloride electrode.

The optional fluid system (200) is any type known in the art that is suitable for performance of the present invention, for example, for dispensing samples onto the working electrode (120), washing the working electrode (120) or adding reagents. The fluid system (200) may provide for continuous flow-through control of the fluid at predetermined rates.

The optional temperature control device (210) is any type known in the art that is suitable for performance of the present invention, for example, for heating or cooling the support (110), the working electrode (120) and/or fluid contacting the support (110) and/or the working electrode (120) as required.

The optional optical scanner or detector (220) is any type known in the art that is suitable for performance of the present invention. The optical scanner or detector (220) may be an ultra violet/visible/near infrared spectrophotometer.

Accordingly, the present invention provides electrode systems for the detection and/or quantification of target nucleic acids. When the nucleic acid capture probe (130) coupled to the working electrode (120) is hybridized with the target nucleic acid (140) to form a nucleic acid complex, and the complex is then intercalated with the threading PIND-Ru-PIND bis-intercalator (150), illumination with a light source at a fixed wavelength results in the generation of a photoelectrochemical response from the intercalated threading PIND-Ru-PIND bis-intercalator (150), thereby producing a photocurrent action spectrum. The application of illumination cycles causes a photocharging and discharging current, proportional to the amount of intercalated threading PIND-Ru-PIND bis-intercalator (150). Measurement of this current therefore provides a measure of the amount of intercalated threading PIND-Ru-PIND bis-intercalator (150), which in turn is a measure of the amount of hybridized target nucleic acid (140), as the threading PIND-Ru-PIND bis-intercalator (150) preferentially intercalates with double-stranded nucleic acid complexes.

The present invention also provides for a biosensor “chip”, otherwise known as an array or microarray. The biosensor chip may comprise a chip, at least one working electrode (120) disposed on the chip, a nucleic acid capture probe (130) coupled to the working electrode (120) and comprising a sequence complimentary to a sequence of a target nucleic acid (140) and a threading PIND-Ru-PIND bis-intercalator (150). Thus, in some embodiments, the working electrode (120) disposed on the support (110) comprises an array, microarray or “chip” onto which is coupled a plurality of nucleic acid capture probes (130). An entire array can be positioned upon a single working electrode (120), or can be positioned across a plurality of working electrodes (120), each of which is disposed upon the support (110). In other embodiments, the array of nucleic acid capture probes (130) is positioned upon one or more working electrodes (120) that are attached to either one surface or a plurality of surfaces of the support (110). The array is positioned upon the working electrode (120) by any suitable method known in the art, for example, by pipette, ink-jet printing, contact printing or photolithography. The array is comprised of at least one element, with each element comprising at least one nucleic acid capture probe (130). The at least one element may be comprised of a plurality of nucleic acid capture probes (130) of the same sequence. The number of elements comprising an array may be any number from 1 to 10⁹ or more. Where a plurality of elements is positioned on the array, the array elements may be spaced apart at a uniform or a variable distance, or a combination thereof. The distance between the centre of each array element can be any distance suitable for performance of the present invention, for example, 100 μm, 10 μm, 1 μm or any other distance. In some embodiments, the array elements are positioned randomly and then the respective location of each array element is determined. The size and shape of the array elements will depend upon the particular application of the present invention, and different sized and shaped elements can be combined into a single array. The surface of the array can be substantially planar or can have features such as depressions or protuberances, and the array elements can be positioned either into the depressions or onto the protuberances. Such depressions can provide a reservoir for solutions into which the array elements are immersed, or such protuberances can facilitate drying of the array elements, as required for the performance of the present invention. For example, elements may be placed in each well of a 96 well plate. In some embodiments, the working electrode (120) and/or the support (110) can include unique identifiers such as indicia, radio frequency tags, integrated devices such as microprocessors, barcodes or other markings in order to identify each of the array elements. The unique identifiers may additionally or alternatively comprise the depressions or protuberances on the surface of the array. Furthermore, the unique identifiers can provide for correct orientation or identification of the working electrode (120) onto which is positioned the array. The unique identifiers can be read directly by the data capture device (160) or by the optical scanner or detector (220). In use, the biosensor chip may comprise an array of nucleic acid capture probes (130) coupled to the working electrode (120), with sequences complimentary to sequences of a target nucleic acid (140), involving the target nucleic acid (140) hybridizing with the nucleic acid capture probe (130) to form a double-stranded nucleic acid complex, the complex then being intercalated with a threading PIND-Ru-PIND bis-intercalator (150), the amount of the intercalated PIND-Ru-PIND (150) being indicative of the amount of target nucleic acid.

The present invention also provides methods for detecting and/or quantifying target nucleic acids, comprising hybridizing a target nucleic acid (140) to a nucleic acid capture probe (130) coupled to a working electrode (120) disposed on a support (110), thereby forming a double-stranded nucleic acid complex, then intercalating the double-stranded nucleic acid complex with a threading PIND-Ru-PIND bis-intercalator (150), then illuminating the working electrode (120) and/or support (110) with light comprising at least one wavelength that photoactivates the PIND-Ru-PIND bis-intercalator (150), then applying a potential to the working electrode (120), then obtaining a photocurrent action spectrum, wherein the amount of the target nucleic acid (140) is determined from the photocurrent action spectrum.

The step of hybridizing the target nucleic acid (140) to the nucleic acid capture probe (130) is dependent upon the complementarity between the respective sequences of the target nucleic acid (140) and the nucleic acid capture probe (130). If the respective sequences are complementary, the target nucleic acid (140) will hybridize to the nucleic acid capture probe (130). If the respective sequences are not complementary, the target nucleic acid (140) will not effectively hybridize to the nucleic acid capture probe (130) in a manner suitable for performance of the present invention. The target nucleic acid (140) may be dissolved in a suitable solvent, for example, water, an organic solvent or an aqueous buffer, or a combination thereof. In some embodiments, the target nucleic acid (140) is hybridized with the nucleic acid probe (130) by way of the fluid system (200) which in some embodiments is controlled by the data capture device (160). In other embodiments, the temperature control device (210) may be used to vary the temperature at which the hybridization step occurs. The temperature control device (210) may be controlled by the by the data capture device (160). The conditions, or stringency, selected during hybridization of the target nucleic acid (140) to the nucleic acid capture probe (130) will depend upon a variety of parameters, including but not limited to the type of nucleic acid involved, the sequences of the target nucleic acid (140) and the nucleic acid capture probe (130) and the solvent into which the target nucleic acid (140) is dissolved. Such conditions are those known by persons skilled in the art. For example, if either the nucleic acid capture probe (130) or the target nucleic acid (140) is double-stranded, the double-stranded form is denatured into single-stranded form prior to hybridization. Optimum hybridization conditions may be determined by the melting temperature (Tm) of both the target nucleic acid (140) and the nucleic acid capture probe (130), which may be calculated by assigning a value of 2° C. for every A or T residue, and 4° C. for every G or C residue present in the target nucleic acid (140) and the nucleic acid capture probe (130). Rates of hybridization may be optimal when carried out using a temperature approximately 20-30° C. below that of the Tm, although this may vary significantly depending on the particular application of the method. The invention may be suitably performed by hybridization of the target nucleic acid (140) to all or some of the nucleotide residues comprising the nucleic acid capture probe (130), such that either one or both the target nucleic acid (140) and/or the nucleic acid capture probe (130) may have single-stranded portions after the hybridization step.

Optionally, after the hybridization step, the support (110) with the working electrode (120) disposed thereupon and the coupled nucleic acid capture probe (130) hybridized to the target nucleic acid (140) is washed so as to remove target nucleic acid (140) that is not completely hybridized to the nucleic acid capture probe (130). Washing may be achieved by any method known to those skilled in the art that effectively removes incompletely hybridized target nucleic acid (140) whilst retaining target nucleic acid (140) that is completely hybridized to the nucleic acid capture probe (130). The washing step can involve modulation of the fluid system (200) and/or the temperature control device (210), either or both of which can be controlled by the data capture device (160), so as to vary the stringency of the washing step as required for the particular application of the method. Stringency can also be varied according to the composition of the wash fluid. Optimization of wash stringency can be achieved by methods known to those skilled in the art, and may include, for example, calculation of nucleic acid length and composition, washing temperature, and salt concentration.

The double-stranded nucleic acid complex formed by hybridization of the target nucleic acid (140) with the nucleic acid capture probe (130) is then intercalated with a threading PIND-Ru-PIND bis-intercalator (150). In some embodiments, the intercalation step is achieved by use of a fluid system (200) which may be controlled by a data capture device (160). In other embodiments, a plurality of threading PIND-Ru-PIND bis-intercalators (150) is used. In still other embodiments, both the target nucleic acid (140) and the threading PIND-Ru-PIND bis-intercalator (150) are applied to the nucleic acid capture probe (130) at the same time. The threading PIND-Ru-PIND bis-intercalator (150) preferentially binds double-stranded nucleic acid complexes but not single stranded nucleic acid. Thus, the threading PIND-Ru-PIND bis-intercalator (150) only binds to regions of nucleic acid where the target nucleic acid (140) has hybridized to the nucleic acid capture probe (130), and does not bind to single-stranded target nucleic acid (140) or single-stranded nucleic acid capture probes (130). In one preferred embodiment, the intercalation of the double-stranded nucleic acid complex with the threading PIND-Ru-PIND bis-intercalator (150) forms a stable adduct by intercalation of two naphthalene diimide groups from the threading PIND-Ru-PIND bis-intercalator (150) with the double-stranded nucleic acid complex, forming an ion pair between a phosphate of the double-stranded nucleic acid complex and a bicationic Ru(bpy)₂²⁺ group of the threading PIND-Ru-PIND bis-intercalator (150). Optimization of the intercalation of the threading PIND-Ru-PIND bis-intercalator (150) will depend upon factors including but not limited to the length of hybridization between the target nucleic acid (140) and the nucleic acid capture probe (130), and the concentration of target nucleic acid (140), nucleic acid capture probe (130) and threading PIND-Ru-PIND bis-intercalator (150). Each of these and potentially other factors can be optimized by those skilled in the art.

After intercalation of the threading PIND-Ru-PIND bis-intercalator (150) with the double-stranded nucleic acid complex, the working electrode (120) is illuminated by the light source (170) which is controlled by the data capture device (160). The illumination may be calibrated so as to excite primarily only the intercalated threading PIND-Ru-PIND bis-intercalator (150) and not the “ground” non-intercalated threading PIND-Ru-PIND bis-intercalator (150). Optimization of illumination will depend upon factors including but not limited to the efficiency of excitation of the threading PIND-Ru-PIND bis-intercalator (150) upon intercalation and the concentration of a sacrificial reductant, if used. In some embodiments, the light source (170) is capable of scanning an array of hybridized nucleic acid complexes intercalated with the threading PIND-Ru-PIND bis-intercalator (150), either by movement of the light source (170) or movement of the working electrode (120). In other embodiments where there is an array, the light source is capable of scanning individual array elements. The potential of the working-electrode (120) is controlled by the data capture device (160), which may bias to a potential capable of oxidizing the excited intercalated threading PIND-Ru-PIND bis-intercalator (150) but not the ground non-intercalated threading PIND-Ru-PIND bis-intercalator (150). In some embodiments, the nucleic acid capture probe (130) is optionally contacted with a sacrificial reductant, which can reduce the oxidized intercalated threading PIND-Ru-PIND bis-intercalator (150) from the excited state to the ground state. The threading PIND-Ru-PIND bis-intercalator (150) can thereby generate a continued photocurrent for the duration of the illumination which can be measured by the data capture device (160). In particular embodiments, the working electrode (120) does not oxidize the sacrificial reductant directly, thus minimizing potentially interfering background currents. The composition of the sacrificial reductant is any known to those skilled in the art that enables performance of the present invention, for example, tertiary amines, ethylenediaminetetraacetic acid, tripropylamine and β-lactam.

The step of obtaining a photocurrent action spectrum of the working electrode involves measurement of the current at the working electrode (120) by any means known to those skilled in the art that is suitable for performance of the present invention. In one embodiment, the current is measured by the data capture device (160)

The present invention also provides for apparatus for detecting and/or quantifying a target nucleic acid. The apparatus may comprise an electrode system (100), a light source (170) as a means for illuminating a working electrode (120) of the electrode system (100), a data capture device (160) as a means for applying a potential to the working electrode (120), and a data capture device (160) as a means for obtaining a photocurrent action spectrum. The apparatus optionally further comprises a data capture device (160) for analyzing the photocurrent action spectrum, a fluid system (200), a temperature control device (210) and an optical or detector (220). Illumination of the working electrode (120) of the electrode system (100) by the means for illuminating the working electrode (120) of the electrode system (100), and application of a potential to the working electrode (120) by the means for applying a potential to the working electrode (120) generates a photocurrent action spectrum which is obtained by the means for obtaining a photocurrent action spectrum. The amount of target nucleic acid can then be determined from the photocurrent action spectrum.

Further improvements in the sensitivity and specificity of the approach are contemplated through the design of a more selective photoreporter, optimizing hybridization conditions, and further minimizing the background caused by non-hybridization-related uptake of the intercalator.

The present invention will now be further described in greater detail by reference to the following specific examples, which should not be construed as in any way limiting the scope of the invention.

### EXAMPLES

### Example 1

### General Methods and Materials

Oligonucleotides. The oligonucleotide sequences used to demonstrate this invention were:

| | |
|---|---|
| 1. | Synthetic target nucleic acid: |

(SEQ ID NO: 1)

| | |
|---|---|
| | 5′-CAT TCC GTA GAA TCC AGG GAA GCG TGT CAC-3′ |
| 2. | Capture probe for synthetic nucleic acid: |

(SEQ ID NO: 2)

| | |
|---|---|
| | 5′-OHC-(CH)₆-A₆-GTG ACA CGC TTC CCT GGA TTC |
| | TAC GGA ATC-3′ |
| 3. | Capture probe for control biosensor. |

(SEQ ID NO: 3)

| | |
|---|---|
| | 5′-OHC-(CH)₆-A₆-CCT CTC GCG AGT CAA CAG AAT |
| | GCT TAA CAT-3′ |
| 4. | Capture probe 1 for TP53; |

(SEQ ID NO: 4)

| | |
|---|---|
| | 5′-OHC-(CH)₆-A₆-ATG GAG GAT TCA CAG TCG GA-3′ |
| 5. | Capture probe 2 for TP53: |

(SEQ ID NO: 5)

| | |
|---|---|
| | 5′-OHC-(CH)₆-A₆-TCA GTC TGA GTC AGG CCC CA-3′ |
| 6. | Capture probe 1 with single mismatched base: |

(SEQ ID NO: 6)

| | |
|---|---|
| | 5′-OHC-(CH)₆-A₆-TCA GAC TGA GTC AGG CCC CA-3′ |
| 7. | Capture probe 2 with single mismatched base: |

(SEQ ID NO: 7)

| | |
|---|---|
| | 5′-OHC-(CH)₆-A₆-TCA GTC TGA GTC ACG CCC CA-3′ |
| 8. | Capture probe 3 with single mismatched base: |

(SEQ ID NO: 8)

| | |
|---|---|
| | 5′-OHC-(CH)₆-A₆-TCA GTC TCA GTC AGG CCC CA-3′ |
| 9. | Capture probe 4 with single mismatched base: |

(SEQ ID NO: 9)

| | |
|---|---|
| | 5′-OHC-(CH)₆-A₆-ATG GTG GAT TCA CAG TCG GA-3′ |
| 10. | Capture probe 5 with single mismatched base: |

(SEQ ID NO: 10)

| | |
|---|---|
| | 5′-OHC-(CH)₆-A₆-ATG GAG GAT TCA CAG TCG GA-3′ |
| 11. | Capture probe 6 with single mismatched base: |

(SEQ ID NO: 11)

5′OHC-(CH)₆-A₆-ATG GAG GAT ACA CAG TCG GA-3′

A₆refers to the polyadenylation sequence: AAAAAA.

Photoreporter: PIND-Ru-PIND was prepared from [Ru(bpy)₂]Cl₂ and PIND as previously described.¹⁰
Apparatus. Electrochemical experiments were carried out using a CH Instruments model 660A electrochemical workstation (CH Instruments, Austin, Tex.). A conventional three-electrode system, consisting of the indium tin oxide (ITO) working electrode (0.20±0.02 cm²), a nonleak Ag/AgCl (3.0 M NaCl) reference electrode (Cypress Systems, Lawrence, Kans.), and a platinum wire counter electrode, was used in all electrochemical measurements. Ultra-violet (UV)-visible spectra were recorded on a V-570 UV/visual (VIS)/near infrared (NIR) spectrophotometer (JASCO Corp., Japan). Measurements of photocurrent were performed with a Fluorolog®-3 spectrofluorometer (Jobin Yvon Inc, Edison, N.J.) in conjunction with a synchronized 660A electrochemical workstation. The discovery mode of the spectrofluorometer was adopted for photocurrent action experiments at 10 nm interval. The intensity of the monochromatic light incident on the ITO electrode was controlled by adjusting both the slit width and the distance to the illuminator. The light intensity at 490 nm was calibrated by a Newport model 841-PE energy and power meter (Newport Corp., Irvine, Calif.). Illumination was performed from the front of the ITO electrode to prevent the absorption of light by the glass substrate. The three electrodes were hosted in a standard 1.0-cm fluorescence cuvette and arranged in such a way that the working electrode faces the illumination window and the other two electrodes are behind the working electrode. All potentials reported in this work were referred to the Ag/AgCl electrode. All experiments were carried out at room temperature, unless, otherwise stated.
BIosensor preparation, hybridization and detection. The pretreatment and silanization of the ITO electrode were performed according to the method of Russell et al³². Oligonucleotide capture probes immobilization was carried as follows: aldehyde modified capture probes were denatured for 10 min at 90° C. and diluted to a concentration of 0.50 μM in 0.10 M pH 6.0 acetate buffer. A 25 μl aliquot of the capture probes solution was dispensed onto the silanized electrode and incubated for 2-3 h at 20° C. in an environmental chamber. After incubation, the electrode was rinsed successively with 0.10% sodium dodecyl sulphate (SDS) and water. The reduction of the imines was carried out by a 5 min incubation of the electrode in a 2.5 mg/ml sodium borohydride solution made of phosphate buffered saline (PBS)/ethanol (3/1). The electrode was then soaked in vigorously stirred hot water (90-95° C.) for 2 min, copiously rinsed with water, and blown dry with a stream of nitrogen. To minimize non-hybridization-related PIND-Ru-PIND uptake and improve the quality and stability of the capture probe coated electrode, the capture probe coated electrode was immersed in an ethanolic solution of 2.0 mg/ml 11-aminoundecanoic acid (AUA) for 3-5 h. Unreacted AUA molecules were rinsed off and the electrode was washed by immersion in a stirred ethanol for 10 min and followed by thorough rinsing with ethanol and water. The surface density of immobilized capture probes, assessed electrochemically using Tarlov's method¹³, was found to be in the range of 5.0-8.0×10⁻¹² mol/cm², which is 20-25% lower than that found at gold electrodes, probably due to a lower capture probe immobilization efficiency via chemical coupling. The hybridization of the target nucleic acid and its photoelectrochemical detection were carried out in three steps. First, the biosensor was placed in the environmental chamber maintained at 50° C. A 25 μl aliquot of hybridization solution containing the target nucleic acid was uniformly spread onto the biosensor. It was then rinsed thoroughly with a blank hybridization solution at 50° C. after 60 min of hybridization. PIND-Ru-PIND was attached to the hybridized target nucleic acid via bis-threading intercalation after 20 min incubation at 25° C. with a 25 μl aliquot of 50-100 μM PIND-Ru-PIND in tris-ethylenediaminetetraacetic acid (TE) buffer (pH 6.0, adjusted with 10 mM HCl). It was then thoroughly rinsed with the pH 6.0 TE buffer. Photocurrent was measured at 0.10 V in 0.10 M NaClO₄.

### Example 2

### Validation of Gone Detection by PIND-Ru-PIND Photoelectrochemistry

The scheme for photoelectrochemical detection of nucleic acid through direct hybridization and formation of the nucleic acid/photoreporter adduct is distinct from but similar to that of electrochemical detection¹⁰. A monolayer of oligonucleotide capture probes was immobilized onto the ITO electrode surface through chemical coupling. The electrode was then sequentially exposed to the target nucleic acid solution and the intercalator solution. Upon illumination, a photoelectrochemical response (photocharging current) was generated in the system, followed by a discharging current when the illumination was turned off. Both the charging and discharging current correlated directly to the target nucleic acid concentration.

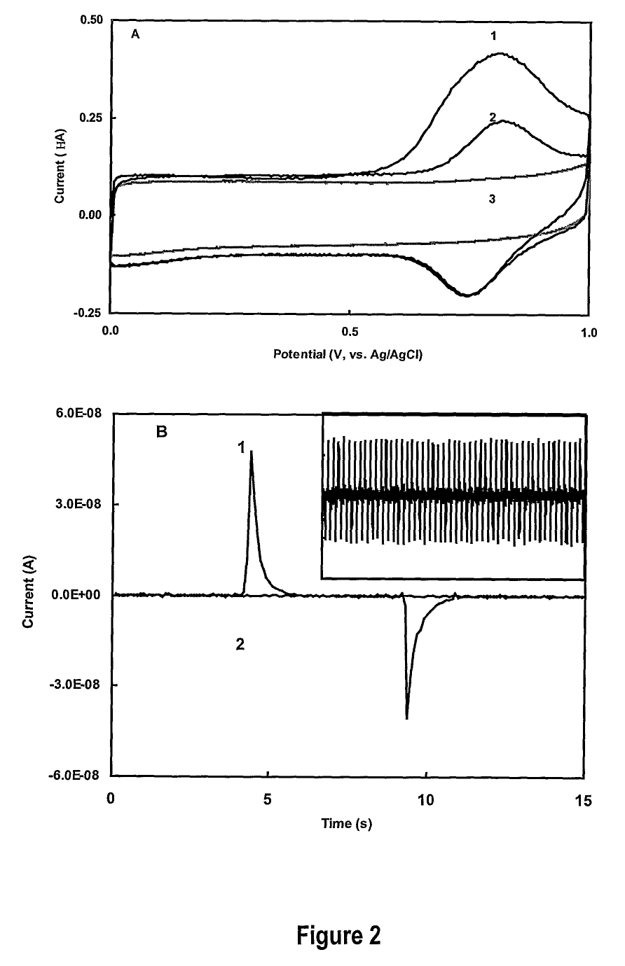

In a first hybridization test, a synthetic oligonucleotide was selected as the target nucleic acid (SEQ ID NO: 1). Upon hybridization at 50,C for 60 min, the target nucleic acid was selectively bound to its complementary capture probe (SEQ ID NO: 2) and became fixed on the biosensor surface. PIND-Ru-PIND was then bound to the biosensor via bis-threading intercalation during subsequent incubation with a 50 μM PIND-Ru-PIND solution. Typical cyclic voltammograms of the biosensor after intercalation are shown in FIG. 2A. As seen in trace 1 in FIG. 2A, considerably higher peak current was observed for the anodic process of the first cycle, indicating that a higher number of electrons was involved in the oxidation process, most probably due to the electrocatalytic oxidation of the captured nucleic acid (guanine bases).¹² The peak current dropped significantly during successive potential cycling and a steady-state voltammogram was attained after 3 cycles between 0 and 1.0 V (FIG. 2A trace 2). Extensive washing and potential cycling thereafter produced no noticeable changes, revealing that the intercalator was robustly bound to the double stranded-DNA (ds-DNA) at the biosensor surface through bis-threading intercalation. FIG. 2A trace 3 shows the voltammogram of a non-complementary capture probe coated electrode (control biosensor) after the same treatments, Negligible redox activity at the redox potential of PIND-Ru-PIND was observed. These results clearly demonstrate that PIND-Ru-PIND selectively interacts with ds-DNA and the resulting ds-DNA/PIND-Ru-PIND adduct has a very slow dissociation rate. Moreover, there was little non-hybridization related PIND-Ru-PIND uptake due to the presence of cationic amine on the biosensor surface.

The high level of stability of the analyte complex and photoreporter adduct may be explained on the basis that after the two naphthalene diimide groups intercalated with the ds-DNA, the bicationic Ru(bpy)₂²⁺ group in PIND-Ru-PIND formed an ion-pair with a phosphate of the ds-DNA, significantly slowing down the dissociation process.

Consequently, the intercalated PIND-Ru-PIND was then evaluated as a potential redox active indicator for electrochemical detection of nucleic acid. A detection limit of 0.50 nM and a dynamic range of 0.8-200 nM were obtained. The hybridization efficiency of ˜32%, estimated at the high end of the dynamic range using the procedure proposed by Tarlov and co-workers¹³, represents ˜10% of the target nucleic acid was actually hybridized, which is comparable to that found in the literature^{10,14,15}. The number of intercalated PIND-Ru-PIND molecules was estimated from the charge under the steady-state voltammogram. Integration of oxidation or reduction current peak at a low scan rate ≦10 mV/s yielded a charge of 0.29 μC, resulting therefore from 3.0 pmol of active and intercalated PIND-Ru-PIND. This number represents <0.1% of PIND-Ru-PIND contained in the assayed droplet and a PIND-Ru-PIND/base pair ratio of 1/5-1/6.

### Example 3

### Photoelectrochemical Characteristics of the Nucleic Acid/PIND-Ru-PIND Adduct

The possibility of utilizing the intercalated PIND-Ru-PIND as a photoreporter for the transduction of nucleic acid hybridization events was examined. Trace 1 in FIG. 28 shows the photoelectrochemical response of the hybridized and PIND-Ru-PIND treated biosensor as the illumination was turned on and off. The photocurrent generation consisted of two steps. An initial spike in photocurrent (photocharging) appeared promptly following the illumination, which was then followed by a quick decay to the background level. It took <2.0 s for the photocurrent to drop to the background level. This represented separations of photogenerated electron-hole pairs at the biosensor surface. Under positive bias, the holes “moved” to the biosensor-solution interface, while electrons “sank” toward the substrate electrode. The quick decay of the photocurrent indicated that a major fraction of the electrons/holes were accumulated at the interface, instead of giving or capturing electrons to or from the electrolyte, respectively.¹⁶

When the light was turned off a similar photocurrent transient behavior of the cathodic current (photodischarging) was observed. The cathodic current decayed with time from an initial maximum down to the background level within 2.0 s, caused by the recombination of the photogenerated electrons and holes at the biosensor surface. The photocharge/discharge cycle was regenerated over 10³ times in 60 min without any noticeable decrease in its intensity. A portion of the regeneration experiment (50 cycles) is depicted in the insert in FIG. 2B. In contrast, the control biosensor failed to capture any target nucleic acid and therefore no photoelectrochemical activity was observed upon illumination (FIG. 2B trace 2). It has been demonstrated that nucleic acid acts as hole-generating biopolymer¹⁷⁻¹⁹. Two limiting mechanisms, namely superexchange²⁰ and discrete hopping²¹, have been proposed. Both mechanisms require a structural distortion of the nucleic acid double helix^{22,23}. The question of how charges migrate over long distance through nucleic acid is still a matter of controversial debate¹⁷⁻²⁶. Nonetheless, it has been generally accepted that the π-stack nucleic acid system has its unique electronic properties, differing significantly from other biopolymers such as proteins and carbohydrates. Charge generation, separation, and recombination processes do take place upon illumination¹⁷⁻²⁶. In a more recent report, photostimulated hole transport through ds-DNA was observed²⁷. It was shown that the efficiency of hole transport is profoundly dependent on the sequence of the DNA and potential bias. The magnitude of the photocurrent decreased sharply when the potential bias became less positive, approaching the background at 0.0 V, leaving only photogenerated charge separation in the system.²⁷

### Example 4

### Analysis of Photocurrent Action Spectra

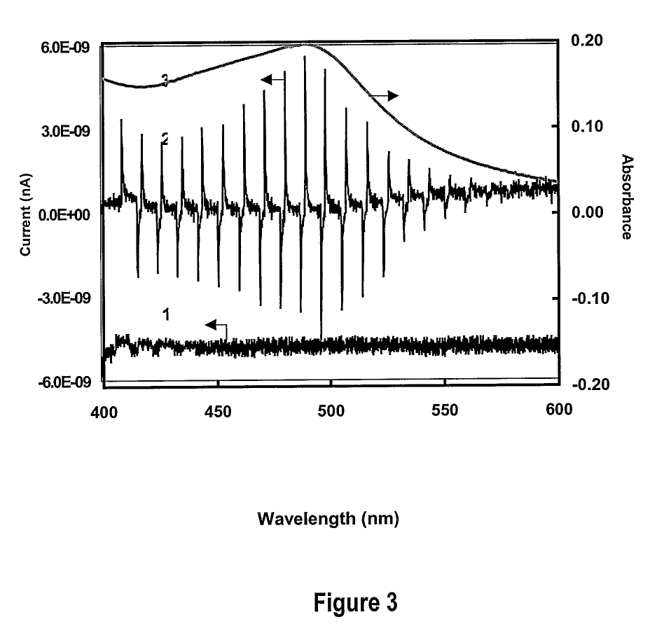

Photocurrent action spectra, i.e. plots of the observed photoelectrochemical responses as a function of the wavelength of the incident light are shown in FIG. 3. It can be seen that the photocurrent action spectrum of the hybridized and intercalated biosensor in the region of 400-600 nm (FIG. 3 trace 2) coincided with the absorption spectrum of PIND-Ru-PIND (FIG. 3 trace 3) with the highest photocurrent at 490 nm, suggesting that PIND-Ru-PIND was the photoactive element on the biosensor surface. This pattern of photocurrent was highly reproducible for numerous on-off illumination cycles. However, in the absence of the intercalated photoreporter, the control biosensor showed little photoactivity (FIG. 3 trace 1). The minute anodic photocurrents observed in the region of 400-440 nm were mainly due to a suppressed photoelectrochemical activity of the substrate electrode.²⁸

### Example 5

### Analysis of Illumination Intensity

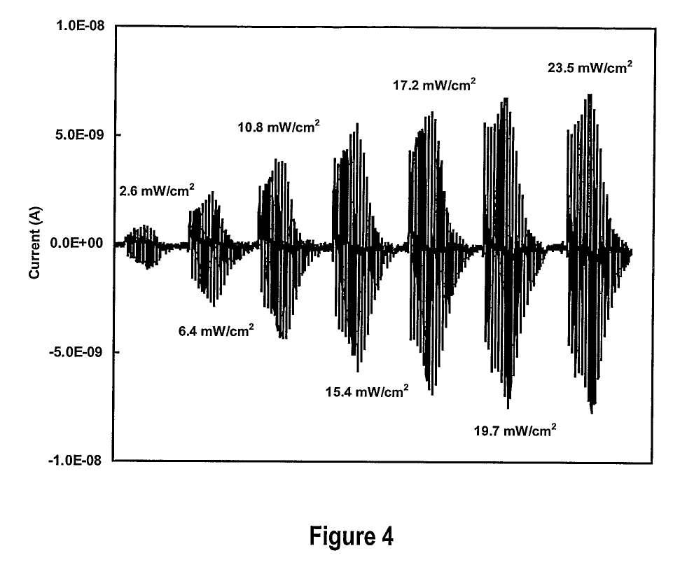

The illumination intensity had a profound effect on the photocurrent. As shown in FIG. 4, the photocharging/discharging current increased linearly with the incident light intensity up to 20 mW/cm² and leveled off at higher intensities. The linear relationship between the photocurrent and the incident light intensity suggested that the photogeneration of charge carriers was a monophotonic process.²⁹

### Example 6

### Analysis of Photocurrent and Applied Potential

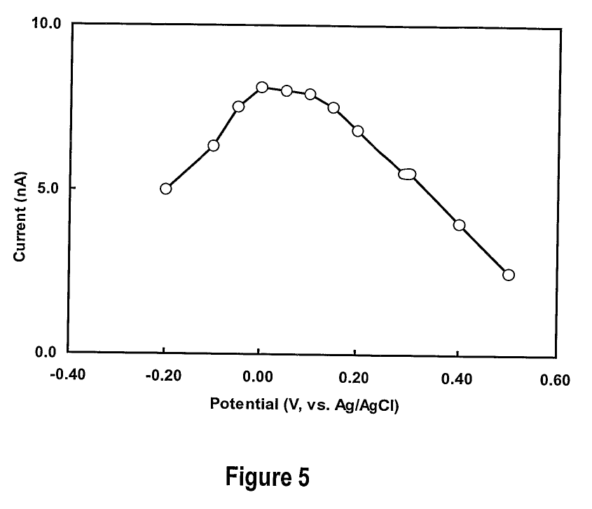

FIG. 5 depicts the dependence of photocurrent on the applied potential. The photocharging current increased gradually when the applied potential moved to the negative direction. The increase in photocharging current with a less positive bias was due to the decrease in charge collection efficiency, as the photogenerated charge carriers were less efficiently transported to the electrode/electrolyte²⁷. The onset potential was found to be ˜0.60 V. Above the onset potential, all photogenerated charge carriers were collected/lost in interfacial reactions. As the applied potential decreased, the fraction of the collected/lost charge carriers decreased. This fraction was practically independent of light intensity, implying that it was only the efficiency with which the charge carriers were transported to or withdrawn from the electrode that limited the photocharging current.

### Example 7

### Analysis of Photocurrent and Illumination Cycle Number

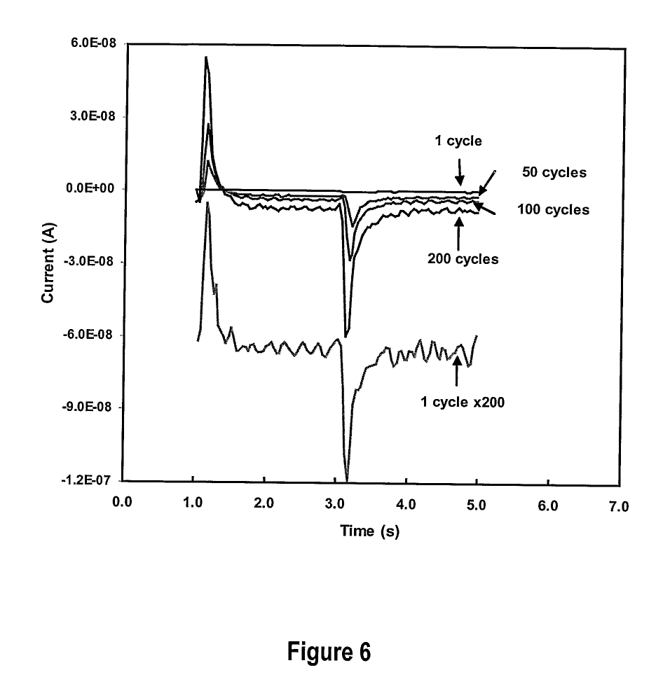

In view of the extremely low dissociation rate of the nucleic acid/photoreporter adduct and the highly reversible photoelectrochemical response, the relationship between photocurrent and illumination cycle number was examined. As demonstrated in FIG. 6, both the photocharging and discharging current increased almost linearly with increasing number of illumination cycles, up to 10³ cycles. Moreover, a substantial reduction of the background noise was also obtained, as random noises tended to cancel out each other after integration over sufficiently large number of cycles.

### Example 8

### Analytical Applications in Nucleic Acid Assays

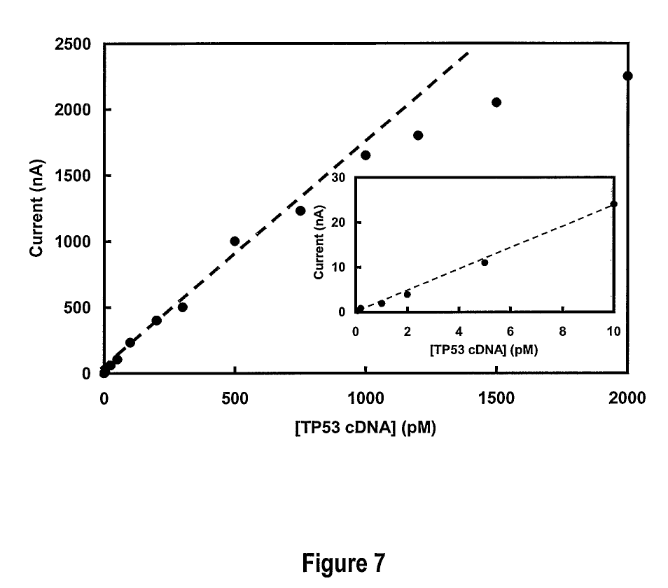

The applicability of the photoelectrochemical approach in nucleic acid assays was tested on genomic samples. A full-length TP53 mouse cDNA was used as the standard and diluted to different concentrations with a pH 8.0 hybridization buffer before use with complementary capture probes (SEQ ID NOs: 4-5). Analyte solutions with different concentrations of cDNA, ranging from 10 fM to 10 nM, were tested. For the control experiment, a non-complementary capture probe was used (SEQ ID NO: 3) in the biosensor preparation. As depicted in FIG. 7, a 1000-cycle integration generated a dynamic range of 50 fM-1.0 nM (R²=0.97) and a detection limit of 20 fM, a 10⁴-fold sensitivity enhancement over the method of voltammetry.

In order to further elucidate the hybridization efficiency and PIND-Ru-PIND loading level, a series of voltammetric measurements were carried out with 1.0 nM TP53 mouse cDNA after hybridization and intercalation. It was shown that ˜3.0 fmoles of TP53 was hybridized, representing ˜0.21% of the surface-bound capture probe and being a much lower value than that for short oligonucleotides (20-50-mers) reported in the prior art.^{8,13-15} In addition, the voltammetric experiments showed that an average of ˜20 PIND-Ru-PIND molecules intercalated with the hybridized TP53. The discrepancy between hybridization efficiency and PIND-Ru-PIND loading level suggest that some of the PIND-Ru-PIND molecules may have intercalated into the secondary structure of TP53^{30,31}, further enhancing the sensitivity of the method.

### Example 9

### Analysis of Biosensor Specificity

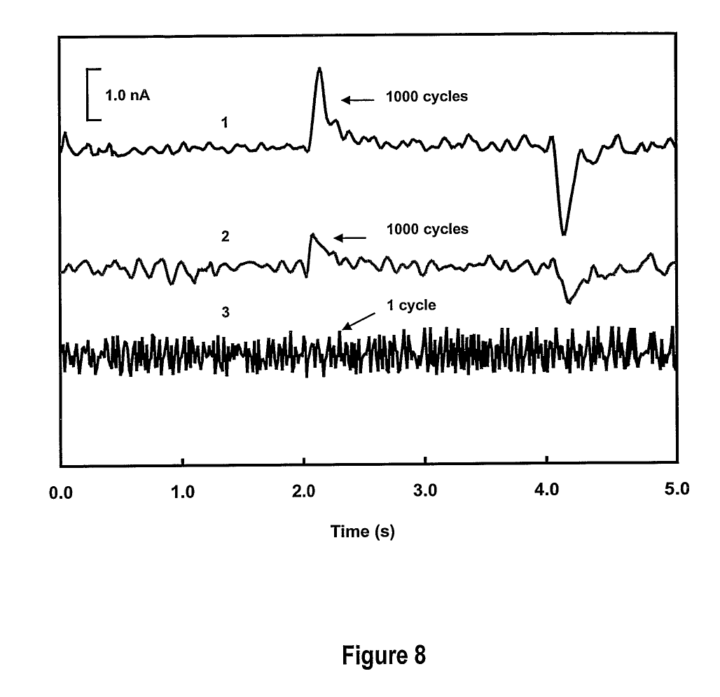

The specificity of the biosensor for detection of target genes was evaluated using a 50 ng mRNA extract by replacing fully complementary capture probes with probes in which one of the bases was mismatched (SEQ ID NOs: 6-11). Considering that there were more than 30,000 genes in this mRNA pool, the actual detectable amount of TP53 was in the range of picograms (˜subpicomolar) on average. As shown in FIG. 8, the current increment for the perfectly matched capture probe coated biosensor was in the range of 2.2-2.6 nA, whereas, for the one base mismatch capture probe coated biosensor, the increment dropped by at least 60% to as low as 0.85 nA, readily allowing discrimination between the perfectly matched and mismatched genes.

### REFERENCES

1. Kricka, L. J, Nonisotopic probing, blotting and sequencing, 2^{nd} Ed. (Academic Press Inc.: San Diego, Calif., 1995).
2. Edwards, M. C. & Gibbs, R. A. Multiplex PCR: Adcantages, development, and applications, PCR-methods and applications. 3, S65-S75(1994).
3. Baugh, L. R., Hill, A. A., Brown, E. L. & Hunter, C. P, Quantitative analysis of mRNA amplification by in vitro transcription. Nucleic Acids Res. 29, e29(2001).
4. Lizardi, P., Huang, X., Zhu, Z., Bray-Ward, P., Thomas, D. & Ward, D. Mutation detection and single molecule counting using isothermal rolling circle amplification. Nature Genet. 19, 225-232(1998).
5. Urdea, M., Running, J., Horn, T., Clyne, J., Ku, L. & Warner, B. A novel method for the rapid detection of specific nucleotide sequences in crude biological samples without blotting or radioactivity; application to the analysis of hepatitis B virus in human serum. Gene 61, 253-264(1987).
6. Wiedom, K. H., Kuhl, H., Galle, J., Caselitz, J. & Volimer, E. Comparison of in situ hybridization, direct and indirect in situ PCR as well as tyramide signal amplification for detection of HPV. Histochem. Cell. Biol. 111, 89-95(1999).
7. Patolsky, F., Ranjit, K. T., Lichtenstein, A. & Willner, I. Dendritic amplification of DNA analysis by oligonucleotide-functionalized Au-nanoparticles. Chem. Commun. 1025-1026(2000).
8. (a), Xie, H., Zhang, C. & Gao, Z. Amperometric detection of nucleic acid at femtomolar levels with a nucleic acid/electrochemical activator bilayer on gold Electrode. Anal. Chem. 76, 1611-1617(2004); (b), Xie, H., Yu, Y. H., Xie, F., Lao, Y. Z. & Gao, Z. A nucleic acid biosensor for gene expression analysis in nanograms of mRNA. Anal. Chem. 76, 4023-4029(2004).
9. Zhang, Y., Kim, H. H. & Heller, A. Enzyme-amplified amperometric detection of 3000 copies of DNA in a 10-μL droplet at 0.5 fM concentration. Anal. Chem. 75, 3267-3269(2003).
10. Tansil, N. C., Xie, H., Xie, F. & Gao. Z. Direct detection of DNA with an electrocatalytic threading intercalator. Anal. Chem. 77, 126-134(2005).
11. (a), Armistead, P. M. & Thorp, H. H. Modification of indium tin oxide electrodes with nucleic acids: Detection of attomole quantities of immobilized DNA by electrocatalysis. Anal. Chem. 72, 3764-3770 (2000); (b), Gore, M. R., Szalai, V. A., Ropp, P. A., Yang, I. V., Silverman, J. S. & Thorp, H. H. Detection of attomole quanitites of DNA targets on gold microelectrodes by electrocatalytic nucleobase oxidation. Anal. Chem. 75, 6586-6592(2003); (c), Napier, M. E., Loomis, C. R., Sistare, M. F., Kim, J., Eckhardt, A. E. & Thorp, H. H. Probing biomolecule recognition with electron transfer: Electrochemical sensors for DNA hybridization. Bioconjugate Chem. 8, 906-913(1997).
12 (a), Mugweru, A. M. & Rusling, J. F. Catalytic square-wave voltammetric detection of DNA with reversible metallopolymer-coated electrodes. Electrochem. Commun. 3, 406-409(2001); (b), Wang, B. & Rusling, J. F. Voltammetric sensor for chemical toxicity using [Ru(bpy)₂poly(4-vinylpyridine)₁₀Cl)]⁺ as catalyst in ultrathin films. DNA damage from methylating agents and an enzyme-generated epoxide. Anal. Chem. 75, 4229-4235(2003); (c), Mugweru, A. & Rusling, J. F. Square wave voltammetric detection of chemical DNA damage with catalytic poly(4-vinylpyridine)-Ru(bpy)₂²⁺ films. Anal. Chem. 74, 4044-4049(2002).
13. Steel, A. B., Herne, T. M. & Tarlov, M. J. Electrochemical quantitation of DNA immobilized on gold. Anal. Chem. 70, 4670-4677(1998).
14. Caruso, F., Rodda, E., Furlong, D. N., Niikura, K.& Okahata, Y. Quartz crystal microbalance study of DNA immobilization and hybridization for nucleic acid sensor development. Anal. Chem. 69, 2043-2049(1997).
15. Satjapipat, M., Sanedrin, R. & Zhou, F. Selective desorption of alkanethiols in mixed self-assembled monolayers for subsequent oligonucleotide attachment and DNA hybridization. Langnuir 17, 7637-7644(2001).
16. Salvador. P. & Gutierrez, C. Analysis of the transient photocurrent-time behaviour of a sintered n-srtio₃ electrode in water photoelectrolysis. J. Electroanal. Chem. 160, 117-130(1984).
17. O'Neill, M. A. & Barton, J. K. Effects of strand and directional asymmetry on base-base coupling and charge transfer in double-helical DNA. J. Am. Chem. Soc. 99, 16543-16550(2002).
18. Wagenknecht, H. A. Reductive electron transfer and transport of excess electrons in DNA. Angew. Chem. Int. Edn. Engi. 42, 2454-2460 (2003).
19. Takada, T., Kawai, K., Cai, X. C., Sugimoto, A., Fujitsuka, M. & Majirna, T. Charge separation in DNA via consecutive adenine hopping. J. Am. Chem. Soc. 126, 1125-1129 (2004).
20. Turro, N. J. & Barton, J. K. Paradigms, supermolecules, electron transfer and chemistry at a distance. What's the problem? The science or the paradigm. J. Biol. Inorg. Chem. 3, 201-209(1998).
21. Giese, B. Long-distance charge transport in DNA: the hopping mechanism. Acc. Chem. Res. 33, 631-636 (2000).
22. Schuster, G. B. Long-range charge transfer in DNA: Transient structural distortions control the distance dependence, Acc. Chem. Res. 33, 253-260 (2000).
23. Wan, C., Fiebig, T., Kelly, S. O., Treadway, G. R., Barton, J. K. & Zewail, A. H. Femtosecond dynamics of DNA-mediated electron transfer, Proc. Natl. Acad. Sci. U.S.A. 96, 6014-6019 (1999).
24. O'Neill, M. A., Dohno, C. & Barton, J. K. Direct chemical evidence for charge transfer between photoexcited 2-aminopurine and guanine in duplex DNA. J. Am. Chem. Soc. 126, 1316-1317 (2004).
25. Lewis, F. D., Letsinger, R. L. & Wasielewski, M. R. Dynamic of photoinduced charge transfer and hole transport in synthetic DNA hairpins. Acc. Chem. Res. 34, 159-170(2001).
26. Beljonne, D., Pourtois, G., Ratner, M. A. & Bredas, J. L. Pathways for photoinduced charge separation in DNA hairpins. J. Am. Chem. Soc. 125, 14510-14517(2003).
27. Okamoto, A., Kamei, T., Tanaka, K. & Saito, I. Photostimulated hole transport through a DNA duplex immobilized on a gold electrode. J. Am. Chem. Soc. 126, 13584-13585(2004).
28. Tansil, N. C., Xie, H. & Gao. Z. Photoelectrochemical behavior of oxalate at an indium tin oxide electrode. J. Phys. Chem. B 108, 16850-16854(2004).
29. Segui, J., Hotchandani, S., Baddou, D, & Leblanc, R. M. Photoelectric properties of ITO CDs chlorophyll-A Ag heterojunction solar cells. J. Phys. Chem. 95, 8807-8812(1992).
30. Eddy, S. R. Non-coding RNA genes and the modern RNA world. Nat. Rev. Genet, 2, 910-929(2001).
31. D'Erchia, A. D., Pesole, G., Tullo, A., Saccone, C & Sbisa, E. Guinea pig p53 mRNA: Identification of new elements in coding and untranslated regions and their functional and evolutionary implications. Genomics, 58, 50-64(1999),

32. Hedges, D. H. P., Richardson, D. J. & Russell, D. A. Electrochemical control of protein monolayers at indium tin oxide surfaces for the reagentless optical biosensing of nitric oxide. Langmuir 20, 1901-1908(2004).

1. An electrode system, wherein the electrode system comprises:
(a) a working electrode;
(b) a nucleic acid capture probe coupled to the working electrode, said probe comprising a sequence complimentary to a sequence of a target nucleic acid; and
(c) a threading PIND-Ru-PIND bis-intercalator such that the target nucleic acid is hybridized to the nucleic acid capture probe to form a double-stranded nucleic acid complex, and the complex is intercalated with the stable intercalator, wherein the amount of the stable intercalator is indicative of the amount of target nucleic acid.

2. The electrode system according to claim 1, wherein the electrode system is capable of detecting the target nucleic acid when the target nucleic acid is used at a concentration up to 1×10⁻¹⁶M.

3. The electrode system according to claim 1, wherein the working electrode comprises an array of nucleic acid capture probes.

4. A method for detecting a target nucleic acid, wherein the method comprises:
(a) hybridizing the target nucleic acid with a nucleic acid capture probe coupled to a working electrode, forming a double-stranded nucleic acid complex;
(b) intercalating the double-stranded nucleic acid complex with a threading PIND-Ru-PIND bis-intercalator;
(c) illuminating the working electrode with light comprising at least one wavelength that photoactivates the stable intercalator;
(d) applying a potential to the working electrode; and
(e) obtaining a photocurrent action spectrum such that the amount of the target nucleic acid is determined from the photocurrent action spectrum.

5. A method for quantifying a target nucleic acid, wherein the method comprises:
(a) hybridizing the target nucleic acid with a nucleic acid capture probe coupled to a working electrode, forming a double-stranded nucleic acid complex;
(b) intercalating the double-stranded nucleic acid complex with a threading PIND-Ru-PIND bis-intercalator;
(c) illuminating the working electrode with light comprising at least one wavelength that photoactivates the stable intercalator;
(d) applying a potential to the working electrode; and
(e) obtaining a photocurrent action spectrum such that the amount of the target nucleic acid is determined from the photocurrent action spectrum.

6. The method according to claim 4 or claim 5, wherein the method is capable of detecting or quantifying the target nucleic acid when the target nucleic acid is used at a concentration in the range up to 1×10⁻¹⁶M.

7. The method according to claim 4 or claim 5, wherein the working electrode comprises an array of nucleic acid capture probes.

8. An apparatus for detecting a target nucleic acid, wherein the apparatus comprises:
(a) the electrode system of claim 1;
(b) means for illuminating the working electrode of the electrode system;
(c) means for applying a potential to the working electrode; and
(d) means for obtaining a photocurrent action spectrum such that in use, illumination of the working electrode by the means for illuminating the working electrode, and application of a potential to the working electrode by the means for applying a potential to the working electrode, generates a photocurrent action spectrum, obtained by the means for obtaining a photocurrent action spectrum.

9. An apparatus for quantifying a target nucleic acid, wherein the apparatus comprises:
(a) the electrode system of claim 1;
(b) means for illuminating the working electrode of the electrode system;
(c) means for applying a potential to the working electrode; and
(d) means for obtaining a photocurrent action spectrum such that in use, illumination of the working electrode by the means for illuminating the working electrode, and application of a potential to the working electrode by the means for applying a potential to the working electrode, generates a photocurrent action spectrum, obtained by the means for obtaining a photocurrent action spectrum.

10. The apparatus according to claim 8 or claim 9, wherein the apparatus is capable of detecting or quantifying the target nucleic acid when the target nucleic acid is used at a concentration in the range up to 1×10⁻¹⁶M.

11. The apparatus according to claim 8 or claim 9, wherein the working electrode of the electrode system comprises an array of nucleic acid capture probes.

12. A biosensor chip, wherein the biosensor chip comprises:
(a) a chip;
(b) at least one wonting electrode disposed on the chip;
(c) a nucleic acid capture probe coupled to the working electrode, the probe comprising a sequence complimentary to a sequence of a target nucleic acid; and
(d) a threading PIND-Ru-PIND bis-intercalator.

13. The biosensor chip according to claim 12, wherein the chip comprises the working electrode.

14. The biosensor chip according to claim 12, wherein the working electrode comprises an array of nucleic acid capture probes.

15. The biosensor chip according to claim 12, wherein the biosensor chip is capable of detecting or quantifying the target nucleic acid when the target nucleic acid is used at a concentration in the range up to 1×10⁻¹⁶M.
